(19) United States
(12) Patent Application Publication
(10) Pub. No.: US 2020/0338282 A1
(43) Pub. Date: Oct. 29, 2020

(54) VAPORIZER CARTRIDGE SYSTEM FOR IDENTIFYING A CARTRIDGE INSERTED INTO A BATTERY UNIT

(71) Applicant: Michael Trzecieski, Toronto, CA (US)

(72) Inventor: Michael Trzecieski, Toronto, CA (US)

(21) Appl. No.: 16/859,887

(22) Filed: Apr. 27, 2020

Related U.S. Application Data
(60) Provisional application No. 62/839,635, filed on Apr. 27, 2019.

(51) Int. Cl. A61M 11/04; A24F 40/42; A24F 40/46; A24F 40/485; A24F 40/51; A24F 40/53; A24F 40/10; A24F 40/44

(52) U.S. Cl. CPC A61M 11/042; A61M 2205/6054; A24F 40/46; A24F 40/485; A24F 40/51; A24F 40/53; A24F 40/10; A24F 40/44; A61M 2205/3334; A61M 2205/52; A61M 2205/0211; A61M 2205/3584; A61M 2205/587; A61M 2206/11; A61M 2205/8206; A24F 40/42

(57) ABSTRACT

A memory module adapter is provided that couples with a conventional 510 cartridge having a cartridge port. The cartridge having a cartridge housing extending from a proximal end of the cartridge to a distal end of the cartridge and an elongated storage compartment formed between the ends, the elongated storage compartment being configured to store a vaporizable material. When the cartridge is inserted into a vaporization device a cartridge coupling port is coupled with the cartridge port which electrically couples the heating assembly with the control circuit assembly and the control circuit assembly type identifier port is electrically coupled with the cartridge type identifier port for a control circuit assembly to serially read type data stored within the at least a two port electrically programmable and electrically readable memory module, wherein the storage compartment, heating assembly and fluid conduit are concentrically disposed. The control circuit assembly for operating in two modes for altering heating profile applied to a heating element assembly based on a type of signal received from cartridge type identifier port.

This application claims the benefit of U.S. Provisional Application No. 62/839,635 filed on Apr. 27, 2019, the entirety of which is incorporated herein by reference.

### FIELD OF THE INVENTION

This application relates generally to vaporization of phyto materials, and more specifically to cartridges usable with vaporizer devices, vaporizer devices using removable cartridges.

### INTRODUCTION

The following is intended to introduce the reader to the detailed description that follows and not to define or limit the claimed subject matter.

Phyto materials extracts are used for various therapeutic and health applications. For instance, cannabis extracts may be used to treat a variety of medical conditions, such as glaucoma, epilepsy, dementia, multiple sclerosis, gastrointestinal disorders and many others. Cannabis extracts have also been used for the general management of pain. These cannabis extracts may be filled into cartridges that are known as 510 cartridges that may then contain a heating and vaporizing system and when heated by a heater they are caused to release an aerosol or vapor which then may be inhaled by a user for therapeutic benefits.

### SUMMARY

The following introduction is provided to introduce the reader to the more detailed description to follow and not to limit or define any claimed or as yet unclaimed invention. One or more inventions may reside in any combination or sub-combination of the elements or process steps disclosed in any part of this document including its claims and figures.

In accordance with one aspect of this disclosure, which may be used alone or in combination with any other aspect, a vaporizer system is provided comprising: a cartridge comprising: a cartridge housing extending from a distal end of the cartridge to a proximal end of the cartridge; an elongated storage compartment, the elongated storage compartment being configured to store a vaporizable material, the elongated storage compartment comprising an inner storage volume wherein the vaporizable material is storable in the inner storage volume, wherein the inner storage volume is enclosed by the cartridge housing; a heating element assembly disposed at a distal end of the storage compartment, the heating element assembly comprising a heating element assembly and a storage interface member, wherein the heating element is in thermal contact with the storage interface member, wherein the storage interface member surrounds the heating element assembly, and the storage interface member includes a plurality of circumferentially spaced fluid apertures fluidly connecting the heating element assembly to the inner storage volume; and a fluid conduit extending through the cartridge housing from a conduit inlet at the distal end to a conduit outlet at the proximal end, wherein the fluid conduit is fluidly connected to the heating element assembly, the fluid conduit passes through a center of the heating assembly from the distal end to the proximal end; an inhalation aperture formed at the proximal end of the fluid conduit; a cartridge port having two electrically insulated electrical contacts electrically coupled with the heating element assembly with the fluid conduit propagating through a center thereof; a device body comprising: a cartridge coupling port for electrically coupling to the heating element assembly through first and second electrical contacts and fluidly coupling of the manifold outlet with the fluid conduit distal end; a control circuit assembly electrically coupled with the cartridge coupling port and comprising a fluid flow sensor fluidly facing the manifold fluid flow path for transmitting an air flow detection signal upon detection of air flow through the air conduit to the control circuit assembly for controllably providing of pulse width modulated electrical power to the heating assembly in dependence upon the air flow detection signal; at least a two port electrically programmable and electrically readable memory module for storing a type date having a first port electrically coupled with one of the first and second electrical contacts of the heating assembly and a second port electrically coupled with a control circuit assembly type identifier port, wherein when the cartridge is inserted into the vaporization device the cartridge coupling port is coupled with the cartridge port which electrically couples the heating assembly with the control circuit assembly and the control circuit assembly type identifier port is electrically coupled with the cartridge type identifier port for the control circuit assembly to serially read type data stored within the at least a two port electrically programmable and electrically readable memory module, wherein the storage compartment, heating assembly and fluid conduit are concentrically disposed; wherein the storage compartment surrounds the heating assembly and the fluid conduit; and wherein the fluid conduit extends along the entire length of the elongated storage compartment from the distal end to the proximal end.

In some embodiments a vaporizer system is provided wherein the device body comprises an air intake manifold comprising an air conduit propagating from an ambient air inlet to a manifold outlet, where the manifold outlet is fluidly coupled with the fluid conduit when the cartridge coupling port is coupled with the cartridge port.

In some embodiments a vaporizer system is provided wherein the device body comprises: a cartridge receptacle formed within the vaporization device, the cartridge receptacle having a proximal end for receiving the cartridge, the cartridge for being inserted into the cartridge receptacle from the proximal end to a distal end thereof wherein the cartridge coupling port is distally disposed within the cartridge receptacle.

In some embodiments a vaporizer system is provided wherein the device body comprises: a cartridge receptacle formed within the vaporization device, the cartridge receptacle having a proximal end for receiving the cartridge, the cartridge for being inserted into the cartridge receptacle from the proximal end to a distal end thereof wherein the cartridge coupling port is distally disposed within the cartridge receptacle and wherein with the cartridge type identifier port is disposed proximally from the cartridge coupling port.

In some embodiments a vaporizer system is provided comprising: a memory module adapter having a proximal coupling port formed a proximal end for being coupled with the cartridge port and a distal coupling port formed at a distal end, the distal coupling port for being coupled with the cartridge coupling port, the memory module adapter comprising the at least a two port electrically programmable and electrically readable memory module.

In some embodiments a vaporizer system is provided wherein the proximal and distal coupling ports comprise a fluid conduit and power coupling for fluidly coupling of the fluid conduit with the manifold outlet and for electrically coupling of the heating element assembly through first and second electrical contacts with the control circuit assembly, and cartridge type identifier port coupled with the second port of the at least a two port electrically programmable and electrically readable memory module, wherein the cartridge type identifier port is electrically coupled with the control circuit assembly type identifier port when the when the cartridge is inserted into the vaporization device receptacle.

In some embodiments a vaporizer system is provided wherein the memory module adapter comprises the at least a two port electrically programmable and electrically readable memory module wherein the at least a two port electrically programmable and electrically readable memory module is for communicating through the cartridge type identifier port and control circuit assembly type identifier port using a 1-wire interface protocol.

In accordance with one aspect of this disclosure, which may be used alone or in combination with any other aspect, a vaporizer system is provided comprising: a cartridge comprising: a cartridge housing extending from a distal end of the cartridge to a proximal end of the cartridge an elongated storage compartment, the elongated storage compartment being configured to store a vaporizable material, the elongated storage compartment comprising an inner storage volume wherein the vaporizable material is storable in the inner storage volume, wherein the inner storage volume is enclosed by the cartridge housing; a heating element assembly disposed at a distal end of the storage compartment, the heating element assembly comprising a heating element assembly and a storage interface member, wherein the heating element is in thermal contact with the storage interface member, wherein the storage interface member surrounds the heating element assembly, and the storage interface member includes a plurality of circumferentially spaced fluid apertures fluidly connecting the heating element assembly to the inner storage volume; and a fluid conduit extending through the cartridge housing from a conduit inlet at the distal end to a conduit outlet at the proximal end, wherein the fluid conduit is fluidly connected to the heating element assembly, the fluid conduit passes through a portion of the heating assembly from the distal end to the proximal end; an inhalation aperture formed at the proximal end of the fluid conduit a cartridge port having two electrically insulated electrical contacts electrically coupled with the heating element assembly; a vaporizer device body comprising: a cartridge receptacle having a proximal end for receiving the cartridge, the cartridge for being inserted into the cartridge receptacle from the proximal end to a distal end thereof; an air intake manifold comprising an air conduit propagating from an ambient air inlet to a manifold outlet; a cartridge coupling port for coupling with the cartridge port for electrically coupling to the heating element assembly through first and second electrical contacts and fluidly coupling of the manifold outlet with the fluid conduit distal end when the cartridge is inserted into the cartridge receptacle; a control circuit assembly electrically coupled with the cartridge coupling port and comprising a fluid flow sensor fluidly facing the manifold fluid flow path for transmitting an air flow detection signal upon detection of air flow through the air conduit to the control circuit assembly for controllably providing of pulse width modulated electrical power to the heating assembly in dependence upon the air flow detection signal; a control circuit assembly type identifier port that is electrically coupled with the control circuit assembly and protrudes in the cartridge receptacle proximate the cartridge coupling port; the control circuit assembly for operating in a first mode of operation wherein when the cartridge is inserted into the cartridge receptacle without a memory module adapter coupled with the cartridge distal end the cartridge coupling electrically couples the heating assembly with the control circuit assembly and the control circuit assembly type identifier port is other than electrically coupled with the cartridge type identifier port and in the second mode of operation with the memory module adapter is secured with the cartridge proximate the distal end, with the memory module adapter disposed between the cartridge coupling port and the cartridge port for the control circuit assembly to serially read data stored within the at least a two port electrically programmable and electrically readable memory module.

In some embodiments a vaporizer system is provided wherein the memory module adapter comprises a proximal coupling port for coupling with the cartridge port and a distal coupling port for coupling with the cartridge coupling port, the proximal and distal coupling ports comprising: a first power and fluid coupling assembly 2004 for fluidly coupling of the air conduit with the fluid conduit and a second electrical coupling for electrically coupling of the second electrical contact as the two electrically insulated electrical contacts electrically coupling of the heating element assembly with the a control circuit assembly.

In some embodiments a vaporizer system is provided wherein the control circuit assembly comprises an energy storage module wherein the control circuit assembly provides pulse width modulated electrical power to the two electrically insulated electrical contacts.

In some embodiments a vaporizer system is provided wherein the second electrical coupling is radially spaced from the first power and fluid coupling assembly which is radially spaced from an air pathway formed through a center of the memory module adapter.

In some embodiments a vaporizer system is provided wherein the proximal coupling port comprises a female threaded end for electrically and fluidly coupling with the cartridge port and a male end disposed at the distal coupling port for releasably coupling with the cartridge coupling port.

In some embodiments a vaporizer system is provided wherein the proximal coupling port is fixedly coupled with the cartridge port and the distal coupling port for releasably coupling with the cartridge coupling port.

In some embodiments a vaporizer system is provided wherein in the first mode of operation wherein when the cartridge is inserted into the cartridge receptacle without a memory module adapter coupled with the cartridge distal end the cartridge coupling electrically couples the heating assembly with the control circuit assembly and the control circuit assembly type identifier port is electrically coupled with the second electrical coupling.

In some embodiments a vaporizer system is provided comprising a two port electrically programmable and electrically readable memory module having a first port electrically coupled with the heating assembly and a second port electrically coupled with a cartridge type identifier port, where data is at least one of read and written to the electrically programmable and electrically readable memory module using a 1-wire interface in the second mode of operation.

In some embodiments a vaporizer system is provided comprising a wicking element disposed between the heating element assembly and the storage interface member, wherein the heating element assembly is in thermal contact with the storage interface member, wherein the storage interface member surrounds the wicking element, and the storage interface member includes the plurality of circumferentially spaced fluid apertures fluidly connecting the wicking element to the inner storage volume.

In some embodiments a vaporizer system is provided wherein the heating element assembly comprises a porous ceramic structure.

In accordance with one aspect of this disclosure, which may be used alone or in combination with any other aspect, a vaporizer system is provided comprising: a memory module adapter comprising a proximal coupling port and a distal coupling port, the memory module adapter for coupling with a cartridge that includes a mouthpiece having an inhalation aperture usable with a vaporizer device, the cartridge having a cartridge housing extending from a proximal end of the cartridge to a distal end of the cartridge and an elongated storage compartment formed between the ends, the elongated storage compartment being configured to store a vaporizable material, the storage compartment comprising an inner storage volume wherein the vaporizable material is storable in the inner storage volume, wherein the inner storage volume is enclosed by the cartridge housing and a heating element assembly disposed at the distal end of the storage compartment, the heating element assembly comprising a heating element and a storage interface member, wherein the heating element is in thermal contact with the storage interface member which surrounds the heating element assembly, and the storage interface member includes at least one fluid apertures fluidly connecting heating element assembly to the inner storage volume, a fluid conduit extending through the housing from a conduit inlet at the first end to a conduit outlet at the second end, wherein the fluid conduit is fluidly connected to the heating element assembly, the fluid conduit passes through the heating element assembly; wherein the fluid conduit extends along approximately the entire length of the elongated storage compartment, a cartridge port for electrically coupling to the heating element assembly through first and second electrical contacts electrically and fluidly coupled with the proximal coupling port and the distal coupling port for releasably coupling with a cartridge coupling port, at least a two port electrically programmable and electrically readable memory module for storing a type date having a first port electrically coupled with one of the first and second electrical contacts of the heating assembly and a second port electrically coupled with a control circuit assembly type identifier port wherein the memory module is for communicating with the control circuit assembly through a serial data communication protocol when the cartridge port is coupled with the memory module adapter and the control circuit assembly.

In some embodiments a vaporizer system is provided comprising: a vaporizer device body comprising: a cartridge receptacle having a proximal end for receiving the cartridge, the cartridge for being inserted into the cartridge receptacle from the proximal end to a distal end thereof; an air intake manifold comprising an air conduit propagating from an ambient air inlet to a manifold outlet; a cartridge coupling port for coupling with the cartridge port for electrically coupling to the heating element assembly through first and second electrical contacts and fluidly coupling of the manifold outlet with the fluid conduit distal end when the cartridge is inserted into the cartridge receptacle; the control circuit assembly electrically coupled with the cartridge coupling port and comprising a fluid flow sensor fluidly facing the manifold fluid flow path for transmitting an air flow detection signal upon detection of air flow through the air conduit to the control circuit assembly for controllably providing of pulse width modulated electrical power to the heating assembly in dependence upon the air flow detection signal the control circuit assembly type identifier port that is electrically coupled with the control circuit assembly and protrudes in the cartridge receptacle proximate the cartridge coupling port.

In some embodiments a vaporizer system is provided a vaporizer system wherein the cartridge port is fixedly coupled with the proximal coupling port and wherein the distal coupling port for releasably coupling with the cartridge coupling port comprises one of a magnetic coupling and a threaded coupling.

### BRIEF DESCRIPTION OF THE DRAWINGS

For a better understanding of the described embodiments and to show more clearly how they may be carried into effect, reference will now be made, byway of example, to the accompanying drawings in which:

FIG. 1A shown therein is an example of a vaporization device in accordance with an embodiment of the invention and a vaporizer cartridge system for identifying a cartridge inserted into a battery unit;

FIG. 1B illustrates a cutaway view of a cartridge and a battery unit;

FIG. 1C illustrates a cutaway view of a cartridge and a memory module adapter and showing a coupling path therebetween;

FIG. 1D illustrates a vaporization device with cover removed and ambient air drawn from an external environment into a manifold fluid flow channel;

FIG. 1E illustrates a cartridge inserted into a base within a cartridge receptacle with an inhalation aperture protruding past a housing of the base;

FIG. 2A illustrates a memory module adapter from a bottom perspective view;

FIG. 2B illustrates a memory module adapter from a top perspective view;

FIG. 2C illustrates a cutaway of a memory module adapter showing a first power and fluid coupling assembly as well as a memory module;

FIG. 2D illustrates a cutaway of a memory module adapter with a first power and fluid coupling assembly removed for clarity;

FIG. 3A illustrates an example of an at least a two port electrically programmable and electrically readable memory module;

FIG. 3B illustrates an exemplary circuit for coupling with a control circuit assembly with a memory module;

FIG. 4A illustrates a magnetic strip sticker that includes magnetic markings affixed to an outside of a cartridge; and,

FIG. 4B illustrates a vaporization device in accordance with another embodiment of the invention for use with a magnetic strip sticker including magnetic markings.

### DETAILED DESCRIPTION

Various apparatuses, methods and compositions are described below to provide an example of an embodiment of each claimed invention. No embodiment described below limits any claimed invention and any claimed invention may cover apparatuses and methods that differ from those described below. The claimed inventions are not limited to apparatuses, methods and compositions having all of the features of any one apparatus, method or composition described below or to features common to multiple or all of the apparatuses, methods or compositions described below. It is possible that an apparatus, method or composition described below is not an embodiment of any claimed invention. Any invention disclosed in an apparatus, method or composition described below that is not claimed in this document may be the subject matter of another protective instrument, for example, a continuing patent application, and the applicant(s), inventor(s) and/or owner(s) do not intend to abandon, disclaim, or dedicate to the public any such invention by its DETAILED DESCRIPTION

Various apparatuses, methods and compositions are described below to provide an example of an embodiment of each claimed invention. No embodiment described below limits any claimed invention and any claimed invention may cover apparatuses and methods that differ from those described below. The claimed inventions are not limited to apparatuses, methods and compositions having all of the features of any one apparatus, method or composition described below or to features common to multiple or all of the apparatuses, methods or compositions described below. It is possible that an apparatus, method or composition described below is not an embodiment of any claimed invention. Any invention disclosed in an apparatus, method or composition described below that is not claimed in this document may be the subject matter of another protective instrument, for example, a continuing patent application, and the applicant(s), inventor(s) and/or owner(s) do not intend to abandon, disclaim, or dedicate to the public any such invention by its disclosure in this document.

Furthermore, it will be appreciated that for simplicity and clarity of illustration, where considered appropriate, reference numerals may be repeated among the figures to indicate corresponding or analogous elements. In addition, numerous specific details are set forth in order to provide a thorough understanding of the example embodiments described herein. However, it will be understood by those of ordinary skill in the art that the example embodiments described herein may be practiced without these specific details. In other instances, well-known methods, procedures, and components have not been described in detail so as not to obscure the example embodiments described herein. Also, the description is not to be considered as limiting the scope of the example embodiments described herein.

The terms “an embodiment,” “embodiment,” “embodiments,” “the embodiment,” “the embodiments,” “one or more embodiments,” “some embodiments,” and “one embodiment” mean “one or more (but not all) embodiments of the present invention(s),” unless expressly specified otherwise.

The terms “including,” “comprising,” and variations thereof mean “including but not limited to,” unless expressly specified otherwise. A listing of items does not imply that any or all of the items are mutually exclusive, unless expressly specified otherwise. The terms “a,” “an,” and “the” mean “one or more,” unless expressly specified otherwise.

Embodiments described herein relate generally to vaporization of vaporizable material, such as phyto materials and phyto material products. Although embodiments are described herein in relation to vaporization of phyto material and phyto material products, it will be understood that other vaporizable materials, such as vaporizable nicotine products and/or synthesized vaporizable compounds, or combinations of vaporizable components may be used. For instance, various vaporizable products containing nicotine or plant derived extracts or oils, such as cannabis extract, CBD or terpene extracts and/or synthesized compounds may be used. Phyto material products may be derived from phyto materials such as the leaves or buds of cannabis plants.

Various methods of vaporizing phyto materials and phyto material products, such as cannabis products, are known. Phyto material is often vaporized by heating the phyto material to a predetermined vaporization temperature. The emitted phyto material vapor may then be inhaled by a user for therapeutic purposes.

Devices that vaporize phyto materials are generally known as vaporizers. In some cases, oils or extracts derived or extracted from the phyto materials may also be vaporized. For cannabis oils or extracts, temperatures in the range of about 450 to 800 degrees Fahrenheit may be applied to vaporize these phyto material products may generate phyto material vapor.

The phyto material vapor may be emitted at a temperature that is uncomfortable for a user to inhale. Accordingly, it may be desirable to cool the vapor prior to inhalation.

Phyto material products, such as oils and extracts, may be generated in batches. The batches may be mixed in a liquid or semi-liquid state. This may facilitate testing of the potency of the phyto material product and provide greater consistency of potency throughout a batch of phyto material product.

Phyto material products, such as oils and extracts may be provided in various liquid, semi-liquid forms. These liquid phyto material products may be stored in a cartridge such as a 510 cartridge that may be used with a vaporizer device.

In some cases, a vaporizable material may be added into a cartridge, and in turn, this cartridge is inserted into a vaporizer. However, it may be quite difficult to fill the cartridges with vaporizable material. Typically, a thin syringe is used to inject very dense oil through a very small applicator tip/orifice into the cartridge.

Vaporization devices that provide for removable cartridges to be vaporized may allow users to adjust the type and/or potency of phyto material products being consumed. A user may insert a cartridge of a particular type into their vaporization device based on the desired therapeutic effect. If a different effect is desired, or the cartridge is spent, the old cartridge may be removed and a new or different cartridge may be inserted for subsequent vaporization. However, it is the tracking of these cartridges that may be important for understanding end user usage patterns.

Vaporization of material from a phyto material cartridge may involve airflow through the phyto material cartridge. However, it may be difficult to ensure consistent airflow through the cartridge as the space available within the vaporization devices limits the space available for a fluid conduit through the cartridge.

Embodiments described herein related generally to methods and devices for vaporizing phyto material, in particular liquids containing phyto material such as medical cannabis. In embodiments discussed herein, examples of vaporization devices or vaporizer devices are described that may be used to vaporize cartridges containing vaporizable products such as liquid phyto material products. The example vaporizer devices may be associated with any suitable type of cartridge containing vaporizable liquid materials that is engageable with the vaporizer devices, such as the example cartridges described herein.

Similarly, in embodiments discussed herein, examples of cartridges that are of a particular type that are usable to store liquid vaporizable materials that are vaporizable using vaporizer devices are described. The example cartridges and type identification of the cartridge may be associated with any suitable type of vaporizer device operable to receive the cartridges, such as the example vaporizer devices described herein.

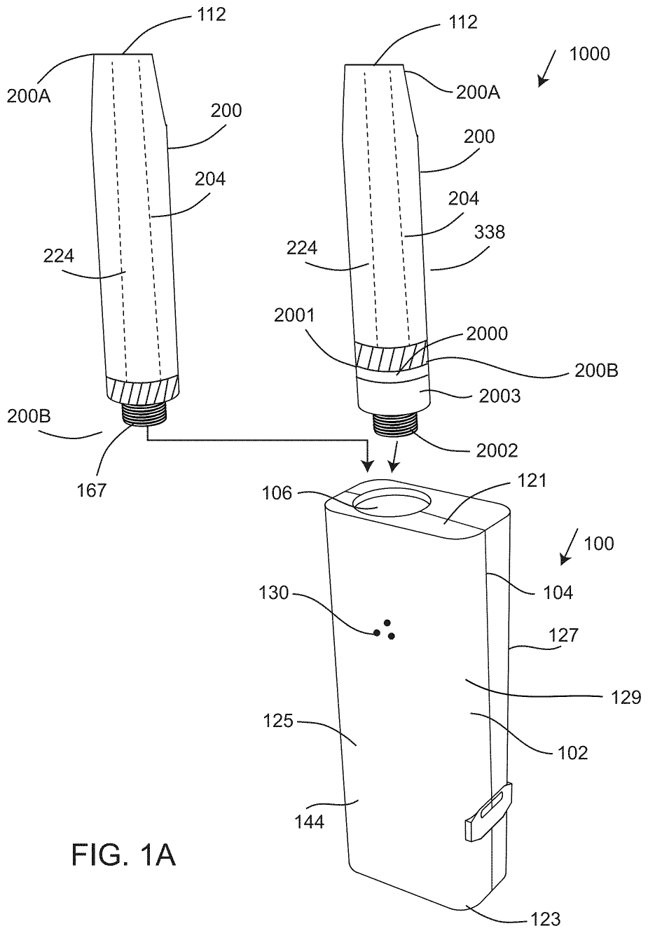

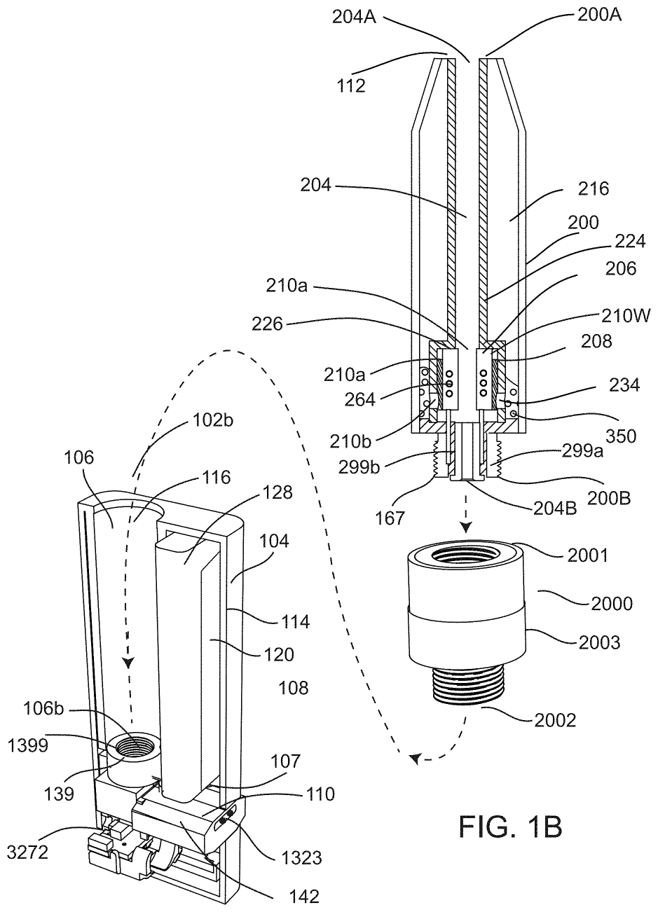

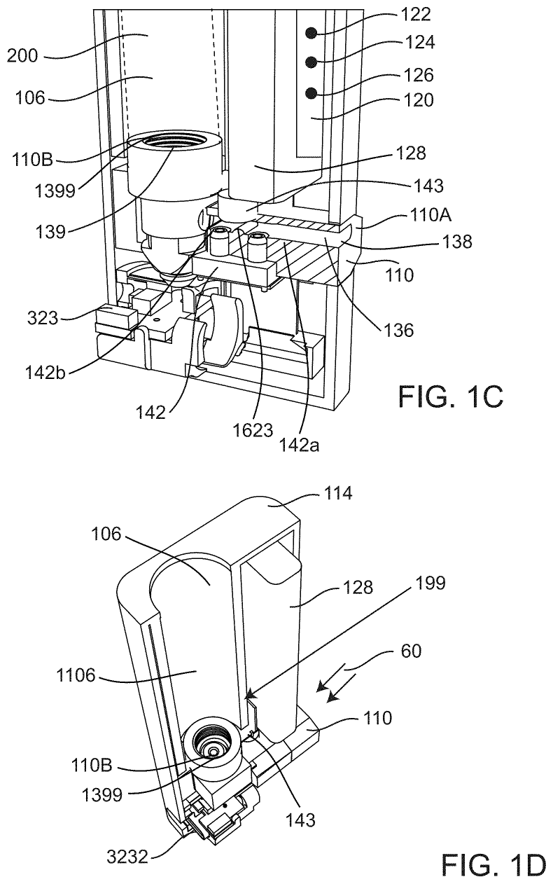

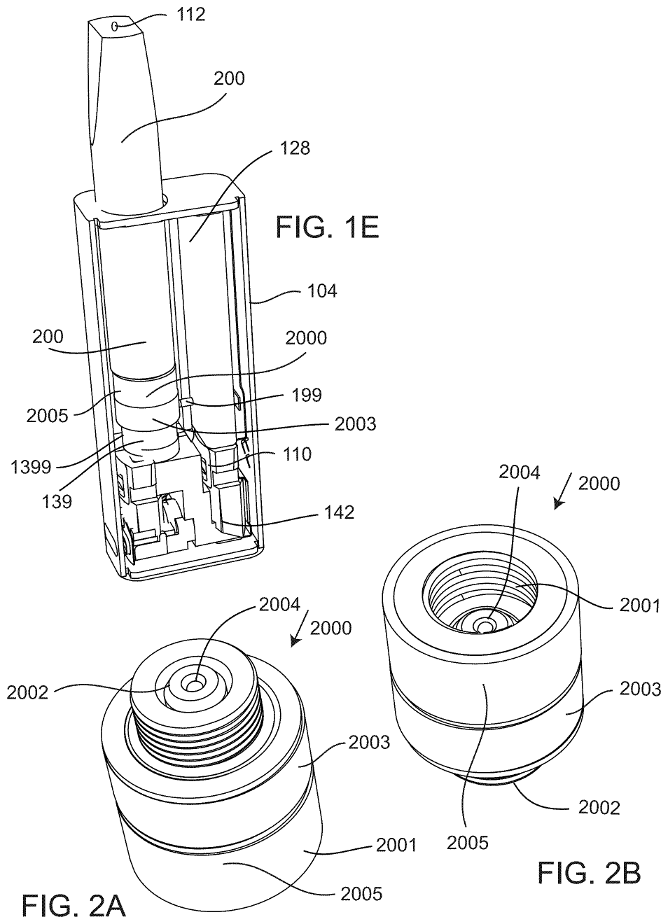

Referring now to FIG. 1A shown therein is an example of a vaporization device 100 in accordance with an embodiment of the invention and a vaporizer cartridge system 1000 for identifying a cartridge inserted into a battery unit.

In the example shown in FIG. 1A, the system 1000 may include a vaporization device 100 that may include a device body 102 that may be formed from two housing parts that include a base 104 and a cover 144. The device body 102 may include a top side or proximal side 121, a bottom side or distal side 123, a front side 125, a rear side 127, and opposed lateral sides 129. Vaporization device 100 generally includes, the front side 125. Base 104 defines opposed lateral sides and the rear side 127 and the bottom side of vaporization device 100. The vaporization device 100 may be used to vaporize material that may be derived from or contain extracts from phyto materials such as extracts derived from cannabis when used with the cartridge 200 inserted therein that may be used to store liquid vaporizable material. Vaporization device 100 may be used to vaporize phyto material products in a liquid or semi-liquid form, which may be referred to herein as vaporizable liquids or liquid vaporizable materials.

Referring to FIG. 1B, the device body 102 and the cartridge 200 are both shown in cutaway views, device body 102 may be used to house and retain various components of the vaporization device 100, such as a control circuit assembly 108, air intake manifold 110, and a cartridge assembly 200.

A cartridge receptacle 106 or receptacle may be defined within the device body 102 and more specifically within at least one of the base 104 and the cover 144. The cartridge receptacle 106 may be shaped to receive and engage a cartridge 200, such as cartridge 200, which is well known in the art. The cartridge receptacle 106 may extend from the proximal side 121 distally towards the distal side 123 and may not protrude past the distal side 123. In FIG. 1B, the cartridge 200 is shown as not being inserted into the cartridge receptacle 106 for clarity. Typically, such a cartridge is about under 11 mm in diameter. And in some cases about 10.2 mm in diameter and may have a length of about 60 mm, details of which will be explained further below.

The receptacle 106 may be defined in the device body 102 may include a portion or section that defines a cartridge receptacle 106 where each of the cover 144 and the base 104 may include the cartridge receptacle 106. In the example shown, the cartridge receptacle 106 is defined by the cartridge receptacle 106 that extend from a base proximal end towards the base distal end device body 102. The cartridge receptacle 106 may be shaped to receive a phyto material cartridge such as cartridge 200 where the cartridge 200 may be in the shape of an elongated cylinder and having a central axis that is approximately parallel with a long axis of the cartridge receptacle 106 within the device body 102 and for cartridge 200 to be sliding along this long axis of the when the cartridge 200 is being inserted into the cartridge receptacle 106 or removed therefrom. The cartridge receptacle having a proximal end 106a for receiving the cartridge, the cartridge for being inserted into the cartridge receptacle from the proximal end 106a to a distal end 106b thereof wherein the cartridge coupling port is distally disposed within the cartridge receptacle 106. The cartridge 200 may be used to store liquid vaporizable material. The cartridge 200 may be removably mounted to the device body 102 within the cartridge receptacle 106 and frictionally or magnetically held therein to facilitate airflow and electrical contact.

Referring to FIG. 1B, a cutaway view of the cartridge 200 is shown. The cartridge 200 may include a heating chamber 206 and a storage compartment 216. A storage interface member 224 may include at least one or a plurality of apertures positioned facing the storage compartment to allow vaporizable material to contact a wicking element 208 for flowing into the heating chamber 206. The cartridge may include a proximal end 200A and a distal end 200B opposite the proximal end 200A. An inhalation aperture 112 may be formed at the proximal end 200A of the cartridge 200. Cartridge housing 202 may extend between a cartridge proximal end 200A and a cartridge distal end 200B opposite the cartridge proximal end 202A. A housing sidewall may extend between the cartridge proximal end 200A and the cartridge distal end 200B.

The fluid conduit may extend through the cartridge housing 202 from the cartridge proximal end 200A to the cartridge distal end 200B. The fluid conduit 204 may include a distal end 204A or upstream inlet at the cartridge distal end 200B and it may also include a cartridge conduit outlet or inhalation aperture 112 downstream and proximally disposed from the distal end 204B or inlet and have a conduit proximal end 204A and proximate the cartridge proximal end 200A.

The storage compartment or reservoir may be used to store vaporizable material for use with a vaporizer 100. The storage compartment may be enclosed by the outer housing sidewall 214. In the example shown, the storage compartment may be parallel to the fluid conduit. That is, the fluid conduit 204 may define a passage that extends parallel to the storage compartment 216 and the fluid conduit 204 may be fluidly and thermally coupled to the heating element assembly 210. The storage compartment and the fluid conduit 204 may be concentrically disposed about a central axis of the conduit 204.

When the cartridge 200 is inserted into the base 104 and more specifically the cartridge receptacle 106 is inserted into the base, the inhalation aperture 112 may protrude past the housing as shown in FIG. 1E. The storage compartment 216 may also be fluidly connected to a heating element assembly 210. The heating assembly may be used to vaporize vaporizable material 350 stored in the storage compartment 216, where the vaporizable material 350 may be drawn from storage compartment 216 and into wicking element 208 that is thermally connected to the heating element assembly 210. Electrical current from an external energy storage member 128 that is external to the cartridge 200 may be directed through heating element assembly 210 when a cartridge coupling port 1399, disposed within the cartridge receptacle 106, is coupled with the cartridge port 167 for electrically coupling to the heating element assembly 210 through first and second electrical contacts and fluidly coupling of the manifold outlet port 139 with the fluid conduit distal end 204B when the cartridge 200 is inserted into the cartridge receptacle 106. The heat emitted by resistive heating element 264 may heat the vaporizable material that is wicked into the heating element assembly 210 to a predetermined vaporization temperature. The heating element assembly 210 may be disposed proximal the distal end of the cartridge 200.

The heating element assembly 210 may also be used with the wicking element 208. The wicking element 208 may at least partially surround the heating element assembly 210. The wicking element 208 may also be arranged coaxially about the heating element assembly 210 and a distal portion of the storage interface member 224 may be oriented coaxially with the heating element assembly 210.

The heating element assembly 210 may be held in place by the storage interface member 224 against the wicking element 208 which is exposed to the vaporizable material from the storage compartment 216 may be drawn to the heating element assembly 210 by wicking element 208. The vaporizable material in the wicking element 208 may then be heated by the heat emitted by a resistive wire that may be embedded within the heating element assembly 210. The storage interface member 224 may surround the heating element assembly 210, and the storage interface member 224 includes a plurality of circumferentially spaced fluid apertures 234 fluidly connecting the heating element assembly 210 to the inner storage volume 216. The heating element assembly 210 and wicking element 208 may be manufactured using a resistive wire embedded in a porous ceramic material. For example, heating element assembly 210 may be manufactured using a porous ceramic and the porous ceramic acts as the wicking element and may obviate a need for a separate wicking element 208.

The heating element assembly 210 may include the resistive heating element 264, which may be in the form of a plurality of resistive heating wire bands may be positioned between the first and second element ends 210A and 210B, e.g. as shown. The resistive bands 264 may be enclosed with an outer wall 210w of the heating element assembly 210. An outer wall of the heating element assembly 210 may be manufactured from a material having limited thermal conductivity, such as a porous ceramic material. The porous ceramic material may initially provide a partial thermal and electrical insulator that allows the resistive heating element 264 to heat up relatively fast due to the low thermal inertia of the heating element assembly 210. The plurality of resistive heating wire bands 264 may be in the form of a coiled wire embedded within the porous ceramic heating element assembly 230. In some cases, the wicking element 208 may be formed integrally with the heating element assembly 210. For example, the heating element assembly 210 may be manufactured from a porous material (e.g. porous ceramics) with pores sized to receive the vaporizable material 350. The pores may also allow the emitted vapor to pass therethrough when resistive heating element 264 is energized, where in some embodiments a 40-50% open porosity with a tortuous pore structure with a pore size ranging from 20 to 90 micron. A resistance of resistive heating element 264 may be about 0.9 Ohms to about 1.7 Ohms.

In embodiments where both heating element assembly 210 and wicking element 208 may be manufactured using porous materials, the pore sizes of the heating element assembly 210 and wick 208 may differ. For instance, the wicking element 208 may have pores with a smaller diameter than the pores of heating element assembly 210. For example, a porous ceramic material used with heating element assembly 210 may be macro-porous having pores with a diameter larger than 50-80 microns. The wicking element 208 may have pores with diameters smaller than 50 microns.

When assembled, the wicking element 208 and the heating element assembly 210 may be positioned concentrically about the heating chamber cavity 226. The heating chamber cavity 226 may be fluidly connected with a fluid conduit 204. Vapor emitted from heating the vaporizable material in wick 208 may then be drawn into fluid conduit 204 through plurality of circumferentially spaced fluid apertures 234 formed within the interface member 224. The fluid conduit 204 propagating from the distal 200B to the proximal 200A ends of the cartridge 200.

Heating element assembly 210 may be positioned within the heating chamber cavity 226 with the wicking element 208 fluidly coupling the fluid conduit 204 to the storage compartment 216. Apertures formed within the distal portion of the interface member 224 may place the wicking element 208 in fluid communication with vaporizable material 350 held in the storage reservoir 216. The vaporizable material 350 may thus be drawn towards the heating element assembly 210 by wicking element 208 or directly into the heating element assembly 210 without the wicking element 208 as is known to those of skill in the art.

When energized, the resistive heating element 264 may heat the heating element assembly 210 and this may cause the porous ceramic to draw vaporizable material drawn into the heating element assembly 210 for being heated by the resistive heating element 264. By heating the vaporizable material 350 to the predetermined vaporization temperature, a phyto material vapor 70 may be emitted into the heating chamber 226 and upon an inhalation from the inhalation aperture, the emitted vapor 70 may flow through the fluid conduit 204 having sidewalls defined by the interface member 224 out towards the cartridge proximal end from the inhalation aperture.

Conventional 510 cartridges 200 as are known to those of skill in the art may have two electrically insulated electrical, an outside of the 510 thread 299a as a ground electrical contact and the air conduit and power port 299b as a positive contact.

When the cartridge 200 is positioned within the cartridge receptacle 116, the vapor may then be inhaled by a user of vaporizer device 100 and when the heating element assembly is energized. The predetermined vaporization temperature may vary depending on user preference and/or the form of the vaporizable material. The vaporization device 100 may then be activated to vaporize the vaporizable liquid in the cartridge 200 and generate phyto material vapor. A user may then inhale the emitted vapor through inhalation aperture 112 to achieve therapeutic effects.

A user may then fully insert the removable cartridge assembly 200 within the cartridge receptacle 106 by sliding the cartridge into the cartridge receptacle and then rotating the cartridge to secure the threads or in some embodiments using a magnetic coupling comprising at least one magnet for securing the cartridge distal end within the cartridge receptacle.

Because state of the art cartridges 200 only have two contact pins, or two electrical conductors that propagate current to its heating element assembly, it may be difficult to determine and differentiate between these cartridges to determine a type of vaporizable material that is stored therein or a type of heating element assembly or a heating profile to apply to the heating element assembly for facilitating an optimal heating profile for the heating element assembly. Advantageously, the vaporization device 100 is operable to work with generic two pin cartridges 200 as described below as well as with other embodiments of the invention as well as to operate with cartridges that feature more than two pins or ports that facilitate determining a type of vaporizable material stored therein.

Referring to FIG. 1E, the cartridge 200 may include an inhalation aperture 112 provided at a proximal end is provided as part of the cartridge 200 and may be provided as part of a mouthpiece that extends outwardly from the outer surface of the end wall of base 104. Proximate the inhalation aperture 112 there may be a mouthpiece may include a removable mouthpiece cover that may be cleaned and/or replaced.

Referring to FIG. 1B and FIG. 1C, the vaporizer 100 may include a control circuit assembly 108. The control circuit assembly 108 may be positioned within the interior device space 106 (see e.g. FIG. 1B). For instance, control circuit assembly 108 may be positioned within the interior device space 106 and proximate the cartridge receptacle 106.

The control circuit assembly 108 or control circuit assembly may be enclosed within the device body 102 and the control circuit assembly 108 may include a control circuit assembly 120, one or more wireless communication modules (122, 124, 126) such as Bluetooth 122, near-field communication (NFC) 124, and Wi-Fi module 126, and the energy storage module 128, such as one or more batteries. The control circuit assembly 120, Bluetooth module 122, NFC module 124, Wi-Fi module 126, and energy storage module 128 may all be mounted on, or supported by, the assembly support base 114. In some embodiments, the assembly support base 114 may include a motherboard that permits electrical communication between all components mounted thereon.

Energy storage module 128 may be electrically coupled to the control circuit assembly 120 and the one or more wireless modules. The control circuit assembly 120 may be electrically coupled to the wireless modules and may be configured to control the operation of the Bluetooth module 122, the NFC module 124 and the Wi-Fi Module 126. The wireless modules may allow firmware installed on vaporizer device 100, such as the control circuit assembly 120, to be updated remotely (e.g. from a central server or through a user application).

Control circuit assembly 120 may be configured to monitor and control various components of vaporization device 100. For example, control circuit assembly 120 may be used to monitor and control the flow of current from energy storage members 128 to the heating element assembly 210.

Control circuit assembly 120 may also be used to provide user interface functionality and user feedback, such as audio or visual outputs. The control circuit assembly 120 may also be used to control the operation of vaporization device 100, such as monitoring device activation and controlling operation of a heating assembly that is onboard vaporization device 100 (including heating elopement assembly provided within removable phyto material cartridges). Control circuit assembly 120 may also monitor the state of various components of vaporization device 100, such as battery discharge levels, a fluid flow sensor 142 activity or other sensor signals, such as potentially a temperature sensor or a sensor used to measure current being provided to the heating element assembly or a gravity sensor, heating element temperature and so forth. Control circuit assembly 120 may also monitor one or more device sensors and feedback indicators, examples of which are described in further detail below.

In some embodiments, energy storage module 128 may be a rechargeable energy storage module, such as a battery or super-capacitor. Vaporization device 100 may include a power supply port (e.g. a USB-port 3232 or magnetic charging port or wireless charging port, such as Qi wireless charging standard) that allows the energy storage module 128 to be recharged. The energy storage module 128 may optionally be removable to allow it to be replaced.

In some embodiments, the vaporization device 100 may include a plurality of device status indicators, such as a plurality of LEDs 130 as shown in FIG. 1A. The status indicators may include various types of status indicators, such as auditory indicators, visual indicators, haptic feedback (e.g. a vibrating motor). The device status indicators may provide a user with information or feedback on various aspects of the vaporization device operation, such as remaining battery capacity, on/off status, mode of operation (e.g., high heat, medium heat, or low heat), temperature of a heating assembly, fill status of a cartridge, presence or absence of a cartridge in cartridge receptacle 116, whether to initiate an inhalation, whether to inhale deeper, whether to stop inhalation and so on.

For example, one or more indicator lights (e.g. Light-emitting diodes) may be provided on the vaporization device 100. The indicator lights may be electrically coupled to the control circuit assembly 120. Accordingly, the control circuit assembly 120 may control the operation of the indicator lights, as the plurality of LEDs 130. The indicator lights may be visible from the exterior of vaporizer device 100, to allow a user to easily identify the status of the vaporizer device 100. In the example shown, the indicator lights may include a plurality light emitting diodes (LEDs) 130.

The vaporizer device 100 may include the cover 144. The cover 144 may be secured to base 104 to enclose components of the vaporizer device 100.

As shown, the cover 144 may be secured to base 104 overlying the cartridge receptacle 106. The cover 144 may thus enclose the support member 114, and associated components mounted thereon. The cover is shown attached in FIG. 1A and removed in FIG. 1B.

Optionally, device cover 144 may be removably mounted to the body device 102. This may permit access to the control circuit assembly 108 for repairs and/or replacement. In other cases, the device cover 144 may be fixed to base 104 with the control circuit assembly 108.

Device cover 144 may or portion of the vaporizer housing or bas 104 may be manufactured of a non-conductive material and the device base 104 may be manufactured from a metal material, such as die casting. This may facilitate communication using the wireless modules disposed within the receptacle 106. In some embodiments, the device cover 144 may be from rubber or thermoplastic materials. The device cover 144 may be manufactured using material with a higher coefficient of friction than device base 104. This may facilitate attaching and removing the device cover 144 from base 104. The cover 144 may also provide a different tactile sense for a user gripping vaporizer device 100. Base 104 may be manufactured using a metallic material. For example, the base 104 may be manufacturing using a machining process, such as a Computer Numerical Control (CNC) machining process. In other cases, the base may be manufacturing using a metal injection molding (MIM) process or a die casting process. In general, however, the base 104 may be formed as a unitary base (i.e. base 104 may have a unitary construction). Alternative materials may also be used for the base 104. Ceramics, such as ceramics containing zirconium oxide, may be used to manufacture base 104. Alternatively, thermoplastic polymer materials may be used to manufacture base 104.

Referring to FIG. 1C, a cutaway view of the air intake manifold 110 is shown that may have a first manifold end 110A and a second manifold end 110B opposite the first manifold end 110B. In the example shown, the air intake manifold 110 may be mounted on the assembly support base 114.

Air intake manifold 110 may include a manifold fluid flow channel 136 defined therethrough. The manifold 110 may include at least one air input aperture 138, which may be referred to as an ambient air inlet or ambient air aperture that is exposed to an outside environment of the device 100 for facilitating incoming ambient airflow. The manifold 110 may also include a manifold outlet port 139 at the second manifold end 110B. The manifold outlet port 139 may be positioned facing the cartridge receptacle 116. The manifold fluid channel 136 may extend between the one or more ambient air inlets 138 and the manifold outlet port 139, defining a fluid passage between the ambient air inlet and the cartridge receptacle 116.

In some embodiments one or more porous screens may be disposed within fluid channel 136, the porous screens may be configured to encourage laminar air flow in the ambient air entering fluid channel 136. The screen or screens may have pores of about 0.1 mm or 0.2 mm or 0.3 mm. The screens may also filter the ambient air to prevent dirt or debris from entering fluid channel 136 and may also provide for laminar flow into and through portions of the fluid flow channel 136. In other cases, a pressure drop element 1623 may be provided within the fluid channel 136 in the case of a differential pressure sensor being used as the fluid flow sensor 142.

Referring to FIGS. 1C, and 1D in some embodiments, the air intake manifold 110 may include the fluid flow sensor 142. The fluid flow sensor 142 may be configured to determine a volume or mass of ambient air 60 being drawn into the manifold fluid flow channel 136 and have its sensing ports fluidly coupled with the fluid flow channel 136. Optionally, an audio microphone may be positioned with the manifold fluid flow channel 136 to determine a volume or mass of airflow passing through the air intake manifold 110. In some embodiments a pressure sensor or a puff sensor may also be utilized to provide a binary indication of airflow through the fluid flow channel 136.

The fluid flow sensor 142 may be electrically coupled to the control circuit assembly 120. In some embodiments. The fluid flow sensor 142 may provide airflow signals to control circuit assembly 120. The control circuit assembly 120 may use the flow signals to determine the air flow through the air intake manifold 110 for controllable application of power to the heating element assembly 210. Based on detected airflow, the control circuit assembly 120 may perform various operations, such as activating/deactivating the heating assembly and/or adjusting a measured temperature of heating element assembly 210.

The cartridge may be inserted into the cartridge receptacle 106 until the distal end 200a of the cartridge 200 engages the air intake manifold 110 at the manifold outlet port 139 at the second manifold end 110B and the cartridge is secured by threaded engagement or magnetic engagement within the cartridge receptacle 106. The cartridge 200 may thus be releasably secured within the cartridge receptacle 106.

Referring to FIG. 1B, the cartridge 200 may have a cartridge port 167 proximate the distal end 200B that is for threading or magnetically engaging into a cartridge coupling port 1399 proximate the manifold outlet port 139 that may have a female 510 thread for engaging the cartridge port 167 having in some embodiments a male threaded end. The cartridge coupling port 1399 may provide an electrical coupling from the control circuit assembly 108 to the heating assembly of the cartridge 200. This electrical coupling may be for example a ground coupling disposed outwardly from a controlled power coupling that is used to controllably apply electrical energy to the heating assembly.

The cartridge port 167 may have two electrically insulated electrical contacts electrically coupled with the heating element assembly 210 with the fluid conduit propagating through a center thereof. The cartridge coupling port 1399 may be for electrically coupling to the heating element assembly 210 through first and second electrical contacts and fluidly coupling of the manifold outlet port 139 with the fluid conduit 204 distal end.

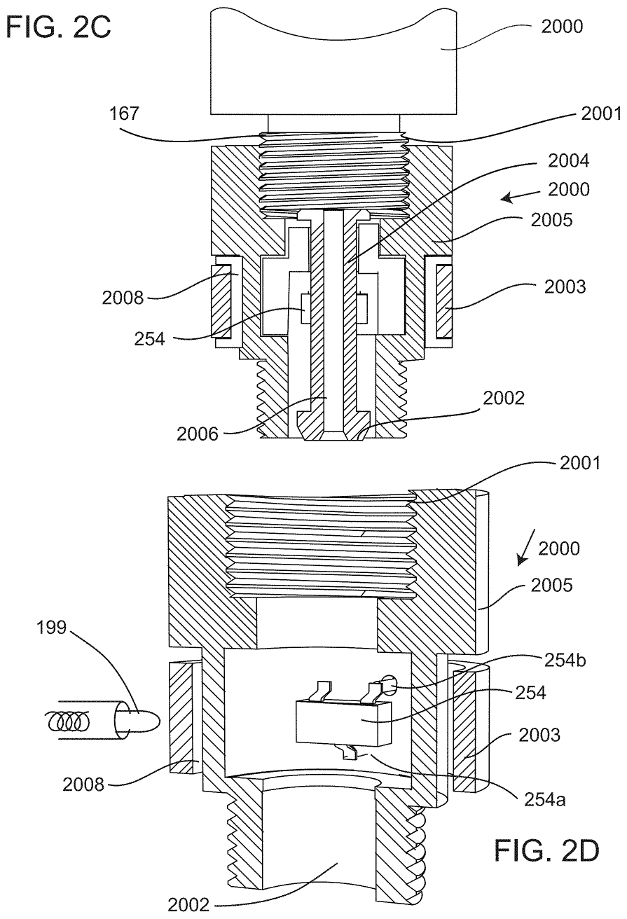

Preferably a memory module adapter (MMA) 2000 may be coupled with the cartridge 200 at the distal end 200B as show in FIG. 1E from a cutaway view of the base unit. An embodiment of the combined cartridge and MMA 338 is shown in FIG. 1A. FIG. 2A illustrates the MMA 2000 from a bottom perspective view and FIG. 2B illustrates the MMA 2000 from a top perspective view. In some embodiments, an outside of the 510 thread 299a, the threaded part is ground while a central and coaxially disposed coaxially within the outside of the 510 thread 299a is the air conduit and power port 299b carries a positive signal that is power modulated in order to provide the controlled power to the heating element assembly, as shown in FIG. 1F.

Referring to FIG. 2A, the MMA 2000 may include a proximal coupling port 2001 for having a female threaded end for electrically and fluidly coupling with the cartridge port 167. The MMA 2000 may also include a distal coupling port 2002 for that may include a male threaded end for electrically and fluidly coupling with the cartridge coupling port 1399 proximate the manifold outlet port 139 that may have a female 510 threaded end.

A first power and fluid coupling assembly 2004 may be for fluidly coupling of the manifold outlet port 139 with the fluid conduit distal end 204 and for electrically coupling with the cartridge air conduit and power port 299b and a second electrical coupling 2005 for electrically coupling of the outside of the 510 thread 299a as the second electrical contact as the two electrically insulated electrical contacts electrically coupling of the heating element assembly with the control circuit assembly 120. The MMA 2000 may also include a cartridge type identifier port 2003 that is electrically insulated from the first and second power couplings using an insulator 2008. The insulator 2008 may be disposed about an outside and radially from the power and fluid coupling assembly 2004 and having a larger diameter. The first power and fluid coupling assembly 2004 electrically couples the air conduit and power port 299b that may propagate a positive electrical signal that is power modulated from the control assembly 128. The insulator 2008 may have a diameter smaller than that of the second electrical coupling 2005, which may have a diameter in at least some aspect of less than or equal to a diameter of the second electrical coupling 2005. In some embodiments at least some of the aforementioned diameters may be less than 11 mm and may be less than 10 mm and in some cases may individually vary between 5 mm and 11 mm.

The second electrical coupling 2005 electrically couples the outside of the 510 thread 299a with the cartridge coupling port 1399 and may be for example a ground coupling or negative electrical coupling. First power and fluid coupling assembly 2004 may be preferably disposed coaxially within the MMA and this coupling may include an air pathway 2006 through a center of the air conduit and power coupling 2004 from the distal to the proximal ends of the MMA 2000. Conventional 510 cartridges 200 as are known to those of skill in the art may have two electrical contacts, an outside of the 510 thread 299a as a ground electrical contact and the air conduit and power port 299b as a positive contact and with the MMA 2000 coupled with the cartridge 200, there may be provided the additional port as the cartridge type identifier port 2003.

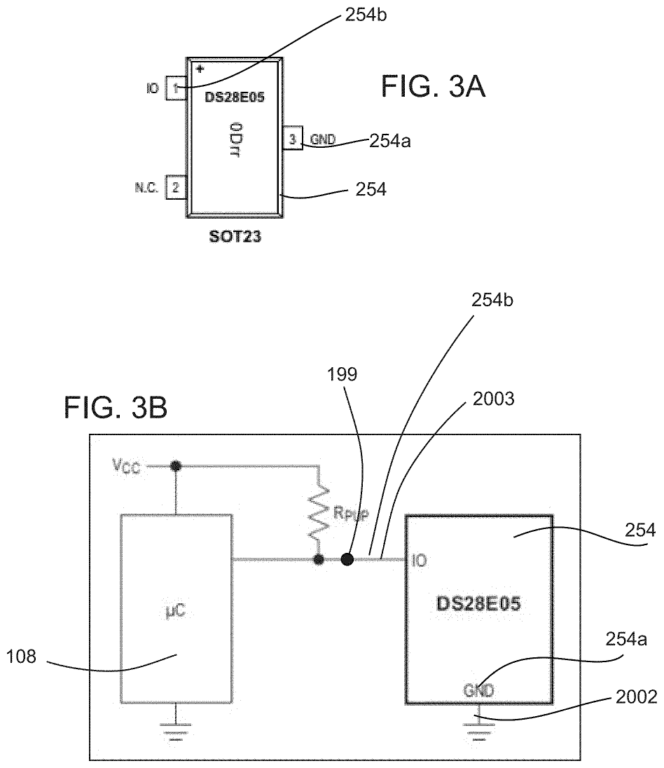

Referring to FIGS. 2D and 3A, the MMA 2000 may include an at least a two port electrically programmable and electrically readable memory module 254 for storing a type date having a first port 254a electrically coupled with one of the first and second electrical contacts of the heating element assembly and a second port 254b electrically coupled with the control circuit assembly type identifier port 199 (FIG. 1E) wherein the memory module is for communicating with the control circuit assembly 120 through a serial data communication protocol when the cartridge port 167 is coupled with the memory module adapter proximal port 2001 and the control circuit assembly is coupled with the distal port 2002.

One of the functions of the MMA 200 is to provide additional data about a type of vaporizable oil and other parameters related thereto, for example terpenes and CBD/THC or other parameters associate with the material for vaporization.

Referring to FIG. 3A, a memory module 254, for example a DS28E05 is shown that is a 112-byte user-programmable EEPROM organized as 7 pages of 16 bytes each and having two ports. The first port 254a is electrically coupled with ground and the second port 254b may be for a 1-wire interface protocol. The 1-wire interface is known generally in the art as a protocol that provides low-speed about 16 kbps data, signaling, and power over a single conductor. Memory pages may be individually set to write protected or EPROM emulation mode through protection byte settings. Each memory module 254 may have its own unique 64-bit ROM identification number (ROM ID) that is factory programmed into the memory module 254. The DS28E05 may communicate over a Maxim single contact 1-Wire® interface as is known in the art. The ROM ID may be used to uniquely identify the MMA 2000 and the 12-byte user-programmable EEPROM may be used to store additional parameters about the type of vaporizable oil or a heating profile to apply to the heating element assembly. Using a two or more-wire interface is also contemplated for the memory module 254, however this may add complexity in the manufacturing of the MMA 2000.

FIG. 2C shows a cutaway of the MMA 2000 to showing a fluid pathway 2006 as well as the memory module 254. Referring to FIG. 2D, first power and fluid coupling assembly 2004 has been removed for clarity to show how the memory module 254 is electrically coupled with the second electrical coupling 2005 and the cartridge type identifier port 2003. Air from the air intake manifold 110 may propagate through the manifold outlet port 139 through first power and fluid coupling assembly 2004 and into the distal end 200B of the cartridge.

The control assembly type identifier port 199 that is electrically coupled with the control circuit assembly 108 is used for electrically coupling with the cartridge type identifier port 2003 and for reading from the memory module 254 using a 1 wire interface protocol for reading at least some of the data stored within the memory module within the ROM ID the user-programmable EEPROM. Other interface protocols may also be used as are known in the art.

Using such a MMA 2000, the control circuit assembly 108 is able to provide an electrical power signal to the cartridge 2000 and also the cartridge when electrically coupled with the MMA 2000 is able to store type information within the memory module 254. This type information may be programmed into the memory module 254 during a cartridge filling process when being filled by a licensed producer.

Referring to FIGS. 1A and 1C, the vaporizer device 100 may operate with either conventional 510 cartridge or with a conventional 510 cartridge that is coupled with the MMA 2000 at the distal end thereof and one that may be integrated with the 510 cartridge where the MMA 2000 proximal port 2001 may be fixedly coupled with the cartridge port 167 and the distal port may be releasably coupled with the cartridge coupling port 1399, such as the combined cartridge and MMA 338 is shown in FIG. 1A. A coupling path is also shown between the cartridge 200 and MMA 2000 and base 100.

The vaporizer device may include device type reading port 199 that may be in the form of a type of spring loaded pogo pin 179 that is electrically coupled with the control circuit assembly 108 spring loaded pogo pin 179 may slide along an outside of the cartridge 200 when it is being inserted into the receptacle 106. The An exemplary circuit for coupling with the control circuit assembly 108 with the memory module 254 is shown in FIG. 3B.

The vaporization device 100 may have the control circuit assembly 120 operate in two modes. In a first mode of operation the vaporization device receives a cartridge 200 within the receptacle 106 that has no MMA 2000 coupled with the distal end 200B of the cartridge. The cartridge type identifier port 2003 in this case is not part of the cartridge 200, as such the control circuit assembly 120 type identifier port 199 may receive a ground signal (as a result of the electrical configuration within the embodiments shown) its electrically coupled with the outside of the 510 thread 299a. Typically a base portion of 510 cartridges is made from metal and this area is electrically connected with the outside of the 510 thread 299a. The control circuit assembly 108 may not be able therefore to read from the non existent memory circuit and the operation of the control circuit assembly 108 may be as is programmed for the first mode of operation, for. However, this port 199 may be spatially oriented within the receptacle 106 such that the port 199 is proximally disposed within the receptacle 106 to electrically couple with a metal housing (ground) of the cartridge 200.

In a second mode of operation, when the MMA 2000 may be electrically coupled with the cartridge and this cartridge 200 and MMA 2000 is inserted into the cartridge receptacle 116, then the cartridge type identifier port 2003 may electrically couple with the control assembly type identifier port 199 and the control circuit assembly 108 is able to read from the memory module 254. or with the cartridge type identifier port 2003.

The memory module 254 may also store temperature related calibration parameters for the resistive wire 164. For example a calibration relationship between a current through the resistive wire and an overall temperature of the heating element assembly 210 may be determined. The determined calibration values may be programmed into the memory module 254 during manufacturing production. The ROM ID may also encode for parameters that are stored on an external server or a cloud server which are then wirelessly downloaded to the control circuit assembly 108.

In the first and second mode the control circuit assembly 120 may be used to control operation of the heating element assembly 210. Cartridge control circuit 242 may be used to activate/deactivate the heating element assembly 210. Cartridge control circuit 242 may also be used to adjust the settings of heating element assembly 210, such as adjusting the predetermined vaporization temperature. In some cases, the predetermined vaporization temperature may be adjusted based on the data stored in the memory module 254 indicating the type of vaporizable material in storage compartment 216. In other embodiments the control circuit assembly 120 may write data back to the memory module 254, for example this data may be related to cartridge usage and inhalation duration or to how many times the cartridge has been inhaled from.

For example, vaporization device may store a calibration lookup table that may be received from the memory module 254 or from a remote server usable to correlate voltage and current being applied to the resistive heating element assembly 210 by the control circuit 120. The temperature of the resistive heating wire 264 may be estimated by sensing a current applied to the heating element assembly 210. In other embodiments through calibration a lookup table may be generated that correlates pulse width modulation (PWM) with a temperature of the heating element assembly 210. In some embodiments, vaporization device may store a calibration lookup table usable to correlate the voltage and current through the resistive heating element 264 with the temperature of heating element assembly 210. The temperature of the resistive heating wire 264 may be estimated by sensing a current applied to the heating element assembly 210.

The current applied to the heating element assembly may be measured by a current sensing integrated circuit, such as ACS722 (manufactured by Allegro MicroSystems) and an analog to digital converter (e.g. a 12, 14 or 16 Bit ADC) to measure battery rail voltage. With the combination of applied current and battery rail voltage, a temperature of the heating element assembly 210 or the resistive heating wire 264 may be extrapolated using a formula based on calibration data contained in a lookup table (LUT).

In the second mode of operation with the data read by the control circuit assembly 120 from the memory module, the heating assembly may be controllably operated in more precise manner than that of the first mode of operation.

Memory module 254 may store data associated with cartridge 200, such as a unique identifier (e.g. an identification serial number) that may be used to identify the removable cartridge assembly 200. The memory 254 may store data (e.g., type, concentration, dose, etc.) regarding the vaporizable material 50 within the removable cartridge assembly 200. In some cases, the unique identifier may be used to retrieve data associated with cartridge assembly 200 and/or vaporizable material 50.

In selecting of the 1-wire interface between the memory module 254 and the control assembly and the control assembly a minimum of ports are utilized as the 1-wire interface supports communication over a single port with a common ground. As a result electrical power from the vaporization device is provided to the cartridge 200 through normal means and as a result the vaporization device is able to function with either a normal cartridge or with the MMA 2000 or with an integrated cartridge that includes the MMA as part of its construction, ie a 3 port cartridge that the is able to also operate using a vaporization device that only provides two signals, ground and control for controlling of the heating element. Magnetic couplings as opposed to threaded couplings are also envisaged to replace the 510 thread and such modifications would be obvious to those skilled in the art. In some embodiments other frictional couplings are also envisaged for electrically and fluidly coupling therebetween.

A vaporization device in accordance with another embodiment of the invention may also be provided where the cartridge includes an upstream end 9000a and a downstream end 9000a and may include a linear barcode sticker 9001 including white and black contrasting markings disposed on a side thereof that propagates between the upstream and downstream ends. A linear optical scanner 9002 that includes an optical transmitter 9002a and an optical received 9002b and a lensing assembly 9002c may be included within the vaporization device 9100 to optically scan within the cartridge receptacle 9116, such that when the cartridge 9000 including the linear barcode 9001 sticker is slid into the cartridge receptacle 9116, the linear optical scanner 9002 reads a difference between white and black contrasting markings in some ways similar to that being provided by the memory module as the identification number, but with less data detail. These detected optical differences may encode for different cartridges. Optionally a microswitch is provided proximate an opening to the cartridge receptacle 9116 to enable and disable operation of the linear optical scanner 9002 to conserve battery power. The control circuit assembly 120 is electrically coupled with the optical scanner and optionally the microswitch.

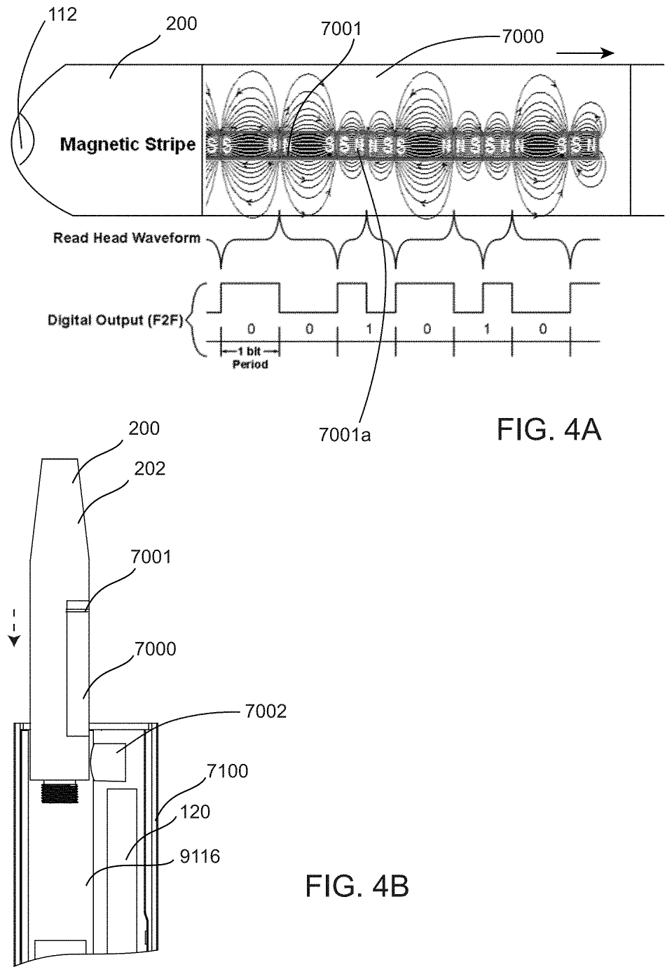

Referring to FIGS. 4A and 4B a vaporization device 7100 in accordance with another embodiment of the invention is shown for use with a cartridge 7000, similar to cartridge 200 may include a magnetic strip sticker 7001 including magnetic markings 7001a disposed on a side thereof as the cartridge 7000. The magnetic markings may be disposed from a proximal end 200A to a distal end 200B thereof. A magnetic head reader 7002 that may be included within the vaporization device 7100 to read from the magnetic strip sticker 7001 within the cartridge receptacle 9116, such that when the cartridge 7000 including the magnetic markings 7001 is slid into the cartridge receptacle 7116, the magnetic head reader 7002 reads a difference between encoded magnetic regions in some ways similar to that being provided by the memory module as type identification and may be used similar to memory module data, but potentially it may be with a lesser amount of data encoded therein. These detected magnetic differences may encode for different cartridge types. Optionally a microswitch is provided proximate an opening to the cartridge receptacle 7116 to enable and disable operation of the magnetic head scanner 7002 to conserve battery power. The control circuit assembly 120 may be electrically coupled with the magnetic head and optionally the microswitch. In some embodiments, the magnetic markings may be formed from light and dark markings that are then read optically by a transmitting and receiving sensor or reflective optical sensor, such that when the cartridge is slid within the receptacle, light and dark differences in light and dark markings, for example executed by a printed sticker disposed onto an outer surface of the cartridge may then be read by the reflective optical sensor. The reflective optical sensor may be electrically coupled with the control circuit assembly 120 and the magnetic head reader 7002 may be electrically coupled with the control circuit assembly 120. The magnetic markings sticker may be adhesively mounted to a side of the cartridge in a position to facilitate magnetic flux communication between the magnetic head reader 7002 and the magnetic strip sticker 7001.

Outer housing of the cartridge may also include a marking or markings (FIGS. 4A and 4B) that may be detected by the vaporizer device 100. For example, the marking(s) may include magnetically or optically scannable barcode located on the outer housing and or label. In some embodiments, the marking(s) may be a pattern, such as a QR code, bar code, etc., that indicate information about the removable cartridge assembly 200 and/or the contents (e.g. vaporizable material 50) within the cartridge removable cartridge 200. In some cases, the marking(s) may be a symbol and/or alphanumeric. The vaporizer device 7100 and cartridge assembly 7000 may also include one or more registration features. The registration features may be configured to ensure that cartridge assembly 7000 is installed in receptacle 9116 in an orientation to facilitate optical and or magnetic scanning of the sticker 7001.

The marking(s) may be “read” or detected directly by the vaporizer device 7100, which may include a camera, scanner or other optical detector (not shown), or it may be indirectly detected via communication with a second device (e.g., a user's smartphone, tablet, etc.) having a camera or an optical detector. For example, the marking(s) on an outer housing of the cartridge 202 and/or label may be detected by the user's smartphone using an application (e.g., software) on the user's smartphone usable to identify characteristics of the cartridge 200. For instance, the application may be configured determine one or more cartridge properties from a lookup table (LUT), or it may directly communicate the marking to the vaporization device 100 that may look up the properties, and/or it may communicate with an external server (not shown) that may look up the properties and communicate them to the vaporizer device 100 directly or through the user's smartphone or Wi-Fi connection. In some embodiments to conserve battery power, the vaporizer device 100 may communicate using a wireless module (e.g. Bluetooth or Wi-Fi radio) when the device 100 is being recharged. In some embodiments, device firmware may be updated while the device 100 is being recharged. The device 100 (i.e. control circuit assembly 120) may be configured to update only while recharging, to prevent unnecessary battery drain.

The cartridge identifier data may include a unique identification number as part of the ID as part of the memory module as represented in 64 bit hexadecimal e.x. “DEAD BEEF 0427 2020”. As part of the EEPROM portion of the memory module, the cartridge identifier data may also include a concentration, such as 10% CBD and 17% THC, or other data related to concentration. The cartridge identifier data may also include a vaporizable material type, such as such as cannabis or nicotine. The cartridge identifier data may also include a fill amount, such as a quantity of vaporizable material 50 that was filled into the storage reservoir, e.g. “500 mg” as shown. The cartridge identifier data may also include a remaining amount 296, such as a quantity of vaporizable material 50 that remains in the storage reservoir 216.

Other cartridge identifier data that may be stored in the memory module 254 may include configuration of the removable cartridge assembly (e.g. electrical properties of heating element assembly), a lot number of the removable cartridge assembly, a date of manufacture of the removable cartridge assembly, an expiration date of the vaporizable material 50, information of the apparatus used to fill the removable cartridge assembly, viscosity properties of the vaporizable material 50, etc. This cartridge identifier data may be directly encoded in the memory module or a reference indicator (e.g. unique identification number) may be provided that the control circuit may use as an index to look up some or all of this information, or a combination of the reference number and the directly encoded cartridge identifier data may be provided.

A filling apparatus may program or encode the cartridge identifier data into the memory module 254 as part of the memory module cartridge MMC 800 after filling the storage reservoir 216 of removable cartridge assembly 200. The MMC 800 is coupled with the cartridge during filling. Optionally the MMC 800 is formed as part of the cartridge as discuss above.

Control circuit assembly 120 as part of control module 108 may be wirelessly coupled with the external server through at least one of the Bluetooth module 122, the NFC module 124 and the Wi-Fi module 126. Accordingly, operating parameters of the control circuit assembly 120 may be adjusted based on the cartridge identifier data stored on the memory module 254 as well as the information/data received from the external server.

The control circuit assembly 120 may adjust the operation of the heating element assembly 210 based on the cartridge identifier data, e.g. adjust the temperature, increase/decrease the power supply from energy storage module 128, etc. Control circuit assembly 120 may also perform calculations based on the mass of air flow entering the vaporization device 100 (e.g. measured by the fluid flow sensor 142 as propagating through the manifold fluid flow channel 136) and the cartridge identifier data to achieve a predetermined operating time for the heating element assembly in relation to a mass or duration of inhalation. The control circuit 110 may also perform calculations based on the mass of air flow entering the vaporization device 100 in conjunction with cartridge identifier data.

Generally, communication between the removable cartridge assembly 200 and the vaporizer device 100 may be one way (e.g., reading information about the removable cartridge assembly 200 and/or the vaporizable material 50 contained in the removable cartridge assembly 200 stored in the memory module 254 by the vaporizer device 100) or it may be two-way (e.g., reading information about the removable cartridge assembly 200 and/or the vaporizable material 50 contained in the removable cartridge assembly 200 and writing information about the operation of the vaporization device 100 into the memory module 254, e.g., number of uses, duration of use, temperature settings, etc.). That is, information may be written in the memory module 254 of removable cartridge assembly 200, and this information may be used to derive other information about the removable cartridge assembly 200, including the amount of material left in the cartridge, etc. The information written in the memory module 254 of removable cartridge assembly 200 may also include air flow data of the mass and/or volume of ambient air 60 passing through the air intake manifold 110 (e.g. collected by fluid flow sensor 142).

In some embodiments, the air intake manifold 110 may include an auditory sensor 143 (FIG. 1C) facing the manifold fluid flow path 136 proximate ambient air inlet and disposed between the between upstream port 142a and downstream port 142b. The auditory sensor 143 may be used to detect air flow into the ambient air inlet 138. The auditory sensor 143 may output a volume signal to the control circuit assembly 120 that may be used to determine whether ambient air 160 is being drawn into the air intake manifold 110. In some cases, the auditory sensor 443 may be configured with a volume threshold. When the volume threshold is reached, the auditory sensor 143 may transmit an air flow detection signal. This signal may be used (as an alternative to, or in combination with signals from mass airflow sensor 142) to wake the control circuit assembly 120 from a low power or sleep mode. In some cases, the auditory sensor 143 may be mounted within the air intake manifold by an insulating material, such as rubber, to reduce false triggers.

The auditory sensor 143 may transmit an air flow detection signal and may replace the mass airflow sensor 142 where a start and stop of airflow is detected with the auditory sensor 143. Additionally, or alternatively, other airflow sensors, such as puff sensors may be used to detect airflow through the air intake manifold 110. For example, signals from the puff sensor may be used to enable/disable operation of a portion of control circuit assembly 120 and/or mass airflow sensor 142. This may ensure that the control circuit assembly 120 and/or mass airflow sensor 142 are not unnecessarily active and draining power from energy storage members 128 in the absence of airflow. In the case of using a convectional cartridge 200, a puff sensor may be used with the airflow through the air intake manifold 110 propagating though the puff sensor or the puff sensor pay be placed in a parallel path with the with the airflow through the air intake manifold 110.

When a user inhales from cartridge inhalation aperture 112, ambient air 60 (FIG. 1D) may be drawn from the external environment into the manifold fluid flow channel 136 via the at least one air input aperture 238. While being drawn by the user's inhalation through the fluid conduit 204, the ambient air 60 may mix with the vapor 70 emitted within the heating chamber conduit section prior to exiting the inhalation aperture 112.

Preferably, user inhalation and the vaporization of the vaporizable material 50 may be synchronized. In some cases, the control circuit assembly 108 may activate the heating element assembly 210 (or provide a signal to cartridge control circuit to activate the heating element assembly 210) in response to the fluid flow sensor 142 detecting ambient air passing through the air intake manifold 110.

Additionally, or alternatively, the plurality of LEDs 130 may indicate that the heating element assembly 210 is heated to the predetermined vaporization temperature. This may indicate that the vaporization device 100 is ready for a user inhalation. In other cases, alternative status indicators may be used. For instance, a vibration notification may be used to notify the user to initiate inhalation, to stop inhalation and/or to increase a depth of inhalation.

Using signals from the airflow sensor 142 and/or auditory sensor 143 to activate the control circuit assembly 120 may allow the vaporization device 100 to conserve energy when the device 100 is not being used. In some cases, the mass airflow sensor 142 may be configured to operate semi-continuously (e.g. at 0.5 Hz, 1 Hz, 2 Hz) in a low power mode to measure a pressure differential between upstream port 142a and downstream port 142b. The lower power mode of mass airflow sensor 142 may be configured to trigger an activation signal to enable/disable operation of a portion of control circuit assembly 120

Advantageously creating a 510 cartridge with the MMA in accordance with the embodiments of the invention adapter allows to add data and tracking to the conventional 510 standard cartridge. This may facilitates being able to track usage frequency and a type of vaporizable material that is being used. Including the 3-pin solution within a convectional 510 cartridge is also advantageously as this may then work within the embodiments of the invention or with conventional 2 pin vaporizer devices already in the market. The MMA 2000 provides for working with existing hardware on the market as well as providing for a proprietary system that allows for operation of the vaporizer device with both the MMA 2000 and without the MMA 2000.

While the above description describes features of example embodiments, it will be appreciated that some features and/or functions of the described embodiments are susceptible to modification without departing from the spirit and principles of operation of the described embodiments. For example, the various characteristics which are described by means of the represented embodiments or examples may be selectively combined with each other. Accordingly, what has been described above is intended to be illustrative of the claimed concept and non-limiting. It will be understood by persons skilled in the art that other variants and modifications may be made without departing from the scope of the invention as defined in the claims appended hereto. The scope of the claims should not be limited by the preferred embodiments and examples, but should be given the broadest interpretation consistent with the description as a whole.

What I claim is:

1. A vaporizer system comprising:
a cartridge comprising:
a cartridge housing extending from a distal end of the cartridge to a proximal end of the cartridge;
an elongated storage compartment, the elongated storage compartment being configured to store a vaporizable material, the elongated storage compartment comprising an inner storage volume wherein the vaporizable material is storable in the inner storage volume, wherein the inner storage volume is enclosed by the cartridge housing;
a heating element assembly disposed at a distal end of the storage compartment, the heating element assembly comprising a heating element assembly and a storage interface member, wherein the heating element is in thermal contact with the storage interface member, wherein the storage interface member surrounds the heating element assembly, and the storage interface member includes a plurality of circumferentially spaced fluid apertures fluidly connecting the heating element assembly to the inner storage volume; and
a fluid conduit extending through the cartridge housing from a conduit inlet at the distal end to a conduit outlet at the proximal end, wherein the fluid conduit is fluidly connected to the heating element assembly, the fluid conduit passes through a center of the heating assembly from the distal end to the proximal end;
an inhalation aperture formed at the proximal end of the fluid conduit;
a cartridge port having two electrically insulated electrical contacts electrically coupled with the heating element assembly with the fluid conduit propagating through a center thereof;
a device body comprising:
a cartridge coupling port for electrically coupling to the heating element assembly through first and second electrical contacts and fluidly coupling of the manifold outlet with the fluid conduit distal end;
a control circuit assembly electrically coupled with the cartridge coupling port and comprising a fluid flow sensor fluidly facing the manifold fluid flow path for transmitting an air flow detection signal upon detection of air flow through the air conduit to the control circuit assembly for controllably providing of pulse width modulated electrical power to the heating assembly in dependence upon the air flow detection signal;
at least a two port electrically programmable and electrically readable memory module for storing a type date having a first port electrically coupled with one of the first and second electrical contacts of the heating assembly and a second port electrically coupled with a control circuit assembly type identifier port,
wherein when the cartridge is inserted into the vaporization device the cartridge coupling port is coupled with the cartridge port which electrically couples the heating assembly with the control circuit assembly and the control circuit assembly type identifier port is electrically coupled with the cartridge type identifier port for the control circuit assembly to serially read type data stored within the at least a two port electrically programmable and electrically readable memory module, wherein the storage compartment, heating assembly and fluid conduit are concentrically disposed;
wherein the storage compartment surrounds the heating assembly and the fluid conduit; and
wherein the fluid conduit extends along the entire length of the elongated storage compartment from the distal end to the proximal end.

2. A vaporizer system according to claim 1 wherein the device body comprises an air intake manifold comprising an air conduit propagating from an ambient air inlet to a manifold outlet, where the manifold outlet is fluidly coupled with the fluid conduit when the cartridge coupling port is coupled with the cartridge port.

3. A vaporizer system according to claim 1 wherein the device body comprises:
a cartridge receptacle formed within the vaporization device, the cartridge receptacle having a proximal end for receiving the cartridge, the cartridge for being inserted into the cartridge receptacle from the proximal end to a distal end thereof wherein the cartridge coupling port is distally disposed within the cartridge receptacle.

4. A vaporizer system according to claim 1 wherein the device body comprises:
a cartridge receptacle formed within the vaporization device, the cartridge receptacle having a proximal end for receiving the cartridge, the cartridge for being inserted into the cartridge receptacle from the proximal end to a distal end thereof wherein the cartridge coupling port is distally disposed within the cartridge receptacle and wherein with the cartridge type identifier port is disposed proximally from the cartridge coupling port.

5. A vaporizer system according to claim 1 comprising:
a memory module adapter having a proximal coupling port formed a proximal end for being coupled with the cartridge port and a distal coupling port formed at a distal end, the distal coupling port for being coupled with the cartridge coupling port, the memory module adapter comprising the at least a two port electrically programmable and electrically readable memory module.

6. A vaporizer system according to claim 5 wherein the proximal and distal coupling ports comprise a fluid conduit and power coupling for fluidly coupling of the fluid conduit with the manifold outlet and for electrically coupling of the heating element assembly through first and second electrical contacts with the control circuit assembly, and
cartridge type identifier port coupled with the second port of the at least a two port electrically programmable and electrically readable memory module, wherein the cartridge type identifier port is electrically coupled with the control circuit assembly type identifier port when the when the cartridge is inserted into the vaporization device receptacle.

7. A vaporizer system according to claim 5 wherein the memory module adapter comprises the at least a two port electrically programmable and electrically readable memory module wherein the at least a two port electrically programmable and electrically readable memory module is for communicating through the cartridge type identifier port and control circuit assembly type identifier port using data, signaling, and power over a single conductor interface protocol.

8. A vaporizer system comprising:
a cartridge comprising:
a cartridge housing extending from a distal end of the cartridge to a proximal end of the cartridge;
an elongated storage compartment, the elongated storage compartment being configured to store a vaporizable material, the elongated storage compartment comprising an inner storage volume wherein the vaporizable material is storable in the inner storage volume, wherein the inner storage volume is enclosed by the cartridge housing;
a heating element assembly disposed at a distal end of the storage compartment, the heating element assembly comprising a heating element assembly and a storage interface member, wherein the heating element is in thermal contact with the storage interface member, wherein the storage interface member surrounds the heating element assembly, and the storage interface member includes a plurality of circumferentially spaced fluid apertures fluidly connecting the heating element assembly to the inner storage volume; and
a fluid conduit extending through the cartridge housing from a conduit inlet at the distal end to a conduit outlet at the proximal end, wherein the fluid conduit is fluidly connected to the heating element assembly, the fluid conduit passes through a portion of the heating assembly from the distal end to the proximal end;
an inhalation aperture formed at the proximal end of the fluid conduit;
a cartridge port having two electrically insulated electrical contacts electrically coupled with the heating element assembly;
a vaporizer device body comprising:
a cartridge receptacle having a proximal end for receiving the cartridge, the cartridge for being inserted into the cartridge receptacle from the proximal end to a distal end thereof;
an air intake manifold comprising an air conduit propagating from an ambient air inlet to a manifold outlet;
a cartridge coupling port for coupling with the cartridge port for electrically coupling to the heating element assembly through first and second electrical contacts and fluidly coupling of the manifold outlet with the fluid conduit distal end when the cartridge is inserted into the cartridge receptacle;
a control circuit assembly electrically coupled with the cartridge coupling port and comprising a fluid flow sensor fluidly facing the manifold fluid flow path for transmitting an air flow detection signal upon detection of air flow through the air conduit to the control circuit assembly for controllably providing of pulse width modulated electrical power to the heating assembly in dependence upon the air flow detection signal;
a control circuit assembly type identifier port that is electrically coupled with the control circuit assembly and protrudes in the cartridge receptacle proximate the cartridge coupling port,
the control circuit assembly for operating in a first mode of operation wherein when the cartridge is inserted into the cartridge receptacle without a memory module adapter coupled with the cartridge distal end the cartridge coupling electrically couples the heating assembly with the control circuit assembly and the control circuit assembly type identifier port is other than electrically coupled with the cartridge type identifier port and in the second mode of operation with the memory module adapter is secured with the cartridge proximate the distal end, with the memory module adapter disposed between the cartridge coupling port and the cartridge port for the control circuit assembly to serially read data stored within the at least a two port electrically programmable and electrically readable memory module.

9. A vaporizer system according to claim 8 wherein the memory module adapter comprises a proximal coupling port for coupling with the cartridge port and a distal coupling port for coupling with the cartridge coupling port, the proximal and distal coupling ports comprising:
a first power and fluid coupling assembly 2004 for fluidly coupling of the air conduit with the fluid conduit and a second electrical coupling for electrically coupling of the second electrical contact as the two electrically insulated electrical contacts electrically coupling of the heating element assembly with the a control circuit assembly.

10. A vaporizer system according to claim 9 wherein the control circuit assembly comprises an energy storage module wherein the control circuit assembly provides pulse width modulated electrical power to the two electrically insulated electrical contacts.

11. A vaporizer system according to claim 9 wherein the second electrical coupling is radially spaced from the first power and fluid coupling assembly 2004 which is radially spaced from an air pathway formed 2006 through a center of the memory module adapter.

12. A vaporizer system according to claim 9 wherein the proximal coupling port 2001 comprises a female threaded end for electrically and fluidly coupling with the cartridge port and a male end disposed at the distal coupling port 2002 for releasably coupling with the cartridge coupling port.

13. A vaporizer system according to claim 9 wherein the proximal coupling port 2001 is fixedly coupled with the cartridge port and the distal coupling port 2002 for releasably coupling with the cartridge coupling port.

14. A vaporizer system according to claim 8 wherein in the first mode of operation wherein when the cartridge is inserted into the cartridge receptacle without a memory module adapter coupled with the cartridge distal end the cartridge coupling electrically couples the heating assembly with the control circuit assembly and the control circuit assembly type identifier port is electrically coupled with the second electrical coupling.

15. A vaporizer system according to claim 8 comprising a two port electrically programmable and electrically readable memory module having a first port electrically coupled with the heating assembly and a second port electrically coupled with a cartridge type identifier port, where data is at least one of read and written to the electrically programmable and electrically readable memory module using a 1-wire interface in the second mode of operation.

16. A vaporizer system according to claim 8 comprising a wicking element disposed between the heating element assembly and the storage interface member, wherein the heating element assembly is in thermal contact with the storage interface member, wherein the storage interface member surrounds the wicking element, and the storage interface member includes the plurality of circumferentially spaced fluid apertures fluidly connecting the wicking element to the inner storage volume.

17. A vaporizer system according to claim 8 wherein the heating element assembly comprises a porous ceramic structure.

18. A vaporizer system comprising:
a memory module adapter comprising a proximal coupling port and a distal coupling port, the memory module adapter for coupling with a cartridge that includes a mouthpiece having an inhalation aperture usable with a vaporizer device,
the cartridge having a cartridge housing extending from a proximal end of the cartridge to a distal end of the cartridge and an elongated storage compartment formed between the ends, the elongated storage compartment being configured to store a vaporizable material, the storage compartment comprising an inner storage volume wherein the vaporizable material is storable in the inner storage volume, wherein the inner storage volume is enclosed by the cartridge housing and a heating element assembly disposed at the distal end of the storage compartment, the heating element assembly comprising a heating element and a storage interface member, wherein the heating element is in thermal contact with the storage interface member which surrounds the heating element assembly, and the storage interface member includes at least one fluid apertures fluidly connecting heating element assembly to the inner storage volume, a fluid conduit extending through the housing from a conduit inlet at the first end to a conduit outlet at the second end, wherein the fluid conduit is fluidly connected to the heating element assembly, the fluid conduit passes through the heating element assembly; wherein the fluid conduit extends along approximately the entire length of the elongated storage compartment, a cartridge port for electrically coupling to the heating element assembly through first and second electrical contacts electrically and fluidly coupled with the proximal coupling port and the distal coupling port for releasably coupling with a cartridge coupling port,
at least a two port electrically programmable and electrically readable memory module for storing a type date having a first port electrically coupled with one of the first and second electrical contacts of the heating assembly and a second port electrically coupled with a control circuit assembly type identifier port wherein the memory module is for communicating with the control circuit assembly through a serial data communication protocol when the cartridge port is coupled with the memory module adapter and the control circuit assembly.

19. A vaporizer system according to claim 18 comprising:
a vaporizer device body comprising:
a cartridge receptacle having a proximal end for receiving the cartridge, the cartridge for being inserted into the cartridge receptacle from the proximal end to a distal end thereof;
an air intake manifold comprising an air conduit propagating from an ambient air inlet to a manifold outlet;
a cartridge coupling port for coupling with the cartridge port for electrically coupling to the heating element assembly through first and second electrical contacts and fluidly coupling of the manifold outlet with the fluid conduit distal end when the cartridge is inserted into the cartridge receptacle,
the control circuit assembly electrically coupled with the cartridge coupling port and comprising a fluid flow sensor fluidly facing the manifold fluid flow path for transmitting an air flow detection signal upon detection of air flow through the air conduit to the control circuit assembly for controllably providing of pulse width modulated electrical power to the heating assembly in dependence upon the air flow detection signal;
the control circuit assembly type identifier port that is electrically coupled with the control circuit assembly and protrudes in the cartridge receptacle proximate the cartridge coupling port.

20. A vaporizer system according to claim 18 wherein the cartridge port is fixedly coupled with the proximal coupling port and wherein the distal coupling port for releasably coupling with the cartridge coupling port comprises one of a magnetic coupling and a threaded coupling.
